(19) United States
(12) Patent Application Publication
(10) Pub. No.: US 2005/0267521 A1
(43) Pub. Date: Dec. 1, 2005

(54) Collagen sponge for arterial sealing

(75) Inventor: Andrew Thomas Forsberg, Minneapolis, MN (US)

Correspondence Address: L. Grant Foster;HOLLAND & HART LLP, P.O. Box 8749, Denver, CO, 80201, US

(73) Assignee: St. Jude Medical Puerto Rico B.V.

(21) Appl. No.: 10/845,495

(22) Filed: May 13, 2004

(51) Int. Cl. A61B 17/08

(52) U.S. Cl. 606/213; 606/155

(57) ABSTRACT

The present invention provides collagen sponge useful in arterial sealing. The collagen sponge is shaped to more closely mimic vascular incisions.

### FIELD OF THE INVENTION

The present invention relates to a vascular closure devices and, more particularly, to a collagen sponge for arterial sealing.

### BACKGROUND OF THE INVENTION

Vascular closure devices are generally known in the art. FIG. 1 shows a conventional vascular closure device 100 useful in closing arterial punctures. Device 100 may include an anchor 102, a bypass tube 104, a hemostatic collagen sponge 106, a suture 108, a carrier tube 110, a tamper tube 112, a device sleeve 114, a reference indicator 116, and a device cap 118.

Using device 100, anchor 102 is deployed to seal a vessel 202 in a patient 204, see FIG. 2. Anchor 102 and collagen sponge 106 seal vessel 202. As shown in FIG. 2, conventionally designed collagen sponge 106 forms a generally conical or spherical shape when fully deployed, which would form a circular cross-section.

Referring now to FIG. 3, a vessel surface 302 is shown. For endovascular procedures, a doctor makes an incision 304 in vessel surface 302. Incision 304 has a length L and a width W. Typically, doctors make incision 304 such that length L is perpendicular to the long axis of vessel 202 and width W is parallel the long axis of vessel 202. This procedure leaves incision 304 with generally an elliptical or oblong shape.

Because the collagen sponge and the incision often have diverse shapes, especially cross-sectional shapes, it would be desirous to provide an improved collagen sponge to assist in sealing incisions.

### SUMMARY OF THE INVENTION

To attain the advantages and in accordance with the purpose of the invention, as embodied and broadly described herein, an improved collagen sponge is provided. The collagen sponge is shaped to more closely match the incision in a vessel to assist in sealing the incision.

The foregoing and other features, utilities and advantages of the invention will be apparent from the following more particular description of a preferred embodiment of the invention as illustrated in the accompanying drawings.

### BRIEF DESCRIPTION OF THE DRAWING

The above and other objects and advantages of the present invention will be apparent upon consideration of the following detailed description, taken in conjunction with the accompanying drawings, in which like reference characters refer to like parts throughout, and in which:

FIG. 1 is a cross-sectional view of a conventional vascular closure device;

FIG. 2 is a cross-sectional view of a collagen sponge deployed about a surgical incision; and

FIG. 3 is a top perspective view of a conventional incision in a vessel;

FIG. 4 is a top cross-sectional view of a collagen sponge deployed over an incision; and

FIG. 5 is a top cross-section view of collagen sponges consistent with the present invention.

### DETAILED DESCRIPTION

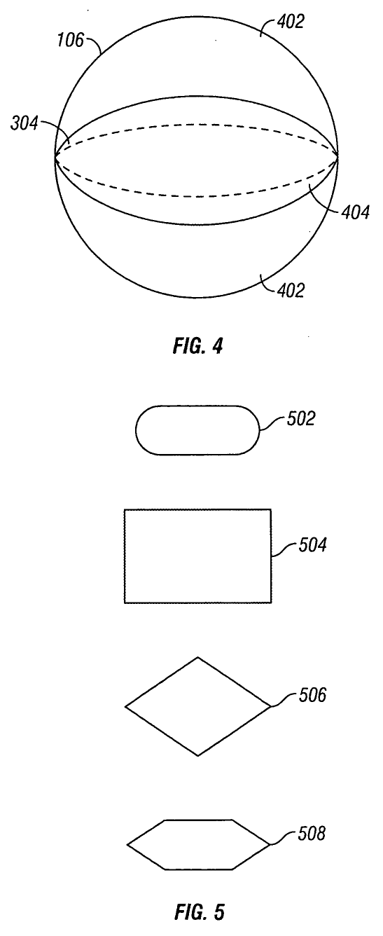

The present invention will now be described with reference to FIGS. 4 and 5. Referring now to FIG. 4, a conventional collagen sponge 106 is shown in cross-section, which cross-section is shown as generally circular although one of ordinary skill in the art would recognize that collagen sponge 106 would not have a smooth circular cross-section. A conventional incision 304 is shown in phantom beneath collagen 106. As shown, collagen sponge 106 has at least one lobe 402 (in this case collagen sponge 106 has 2 lobes 402) in which a majority of the collagen sponge associated with the lobe is not sufficiently adjacent incision 304 to assist in sealing. Lobe 404, however, is sufficiently adjacent incision 304 to assist in sealing incision 304. In other words, lobes 402 of collagen sponge 106 are wasted and unnecessary.

Referring to FIG. 5, collagen sponges consistent with the present invention are shown. In particular, collagen sponge 502 shows collagen sponge 106 with lobes 402 removed. Collagen sponge 502 has a length L′ and a width W′ and generally is formed to mimic the vascular incision. Length L′ and width W′ could be identical to incision length L and width W, but generally collagen sponge 502 is longer and wider than incision 304 after wetting to allow for proper sealing. Collagen sponge 502 could be the same length and width or shorter and narrower, but seepage may occur in these cases. One of skill in the art would recognize on reading the disclosure that collagen sponges can take many shapes where the length to width ratio are not equal, such as for a square or circular shape. For example, the collagen sponge could be have a generally elliptical or oval shape, such as collagen sponge 502, rectangular shape, such as collagen sponge 504, diamond shape, such as collagen sponge 506, hexagon shape, such as collagen sponge 508, or the like. Further, while not shown, collagen sponge 502 could be specially designed for irregular incisions and not have any predefined shape.

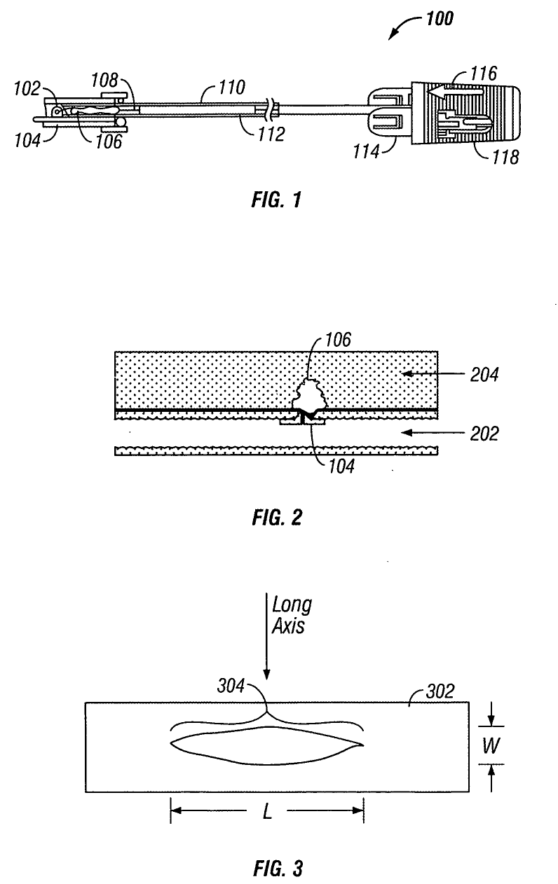

While collagens consistent with the present invention could be deployed using a conventional vascular closure device, as shown in FIG. 1, care would need to be taken to ensure collagen sponge 502, for example, is deployed to match incision 304. In other words, collagen sponge 502's length L′ needs to substantially align with incision 304's length L. Alternative to using delivery conventional devices, such as device 100 above, a modified delivery device could be used. Generally, the only modification to a conventional delivery device would be to alter the device cross-section from generally circular to a shape consistent with the collagen sponge's shape, such as an oval cross-section to match collagen sponge 502 or a rectangular cross-section to match collagesn sponge 504, etc. It is believed a closure device having an oval cross-section would likely accommodate many of the collagen sponges consistent with the present invention.

While the invention has been particularly shown and described with reference to embodiments thereof, it will be understood by those skilled in the art that various other changes in the form and details may be made without departing from the spirit and scope of the invention.

1. An arterial sealing device, comprising:
at least one collagen sponge;
the at least one collagen sponge comprising at least one cross-section; and
the at least one cross-section adapted to mimic a shape of a vascular incision.

2. The device according to claim 1, wherein the at least one cross-section comprises:
a length; and
a width, wherein
the length is greater than the width.

3. The device according to claim 1, wherein the at least one cross-section comprises:
a length; and
a width, wherein
the width is greater than the length.

4. The device according to claim 1, wherein the at least one cross-section comprises at least one of an elliptical shape, an oval shape, a rectangular shape, a triangle shape, a quadrilateral shape, a pentagon shape, a hexagon shape, a heptagon shape, a octagon shape, an enneagon, a decagon shape, and a polygon comprising at least eleven sides.

5. The device according to claim 1, wherein the at least one cross-section comprises an irregular shape.

6. The device according to claim 1, wherein the at least one cross-section comprises a surface area sufficiently greater than a surface area of the incision to inhibit seepage.

7. A vascular closure device comprising an anchor, an improved collagen sponge, at least one suture, and means for delivering the device to a vascular incision, the improved collagen sponge comprising:
a cross-sectional shape such that the length to width ratio of the cross-sectional shape is not 1.

8. The vascular closure device according to claim 7, wherein the cross-sectional shape comprises a polygon.

9. The vascular closure device according to claim 8, wherein the polygon comprises at least one of a triangle shape, a quadrilateral shape, a pentagon shape, a hexagon shape, a heptagon shape, a octagon shape, an enneagon, and a decagon shape.

10. The vascular closure device according to claim 8, further comprising a non-circular means for delivery.

11. The vascular closure device according to claim 10, wherein the non-circular means for delivery comprises a non-circular carrier tube.

12. A vascular closure device, comprising:
an anchor;
a collagen sponge having a non-circular cross-section;
a carrier tube having a non-circular cross-section; and
a suture.

13. The closure device according to claim 12, wherein the collagen sponge non-circular cross-section comprises at least one of an elliptical shape, an oval shape, a rectangular shape, a triangle shape, a quadrilateral shape, a pentagon shape, a hexagon shape, a heptagon shape, a octagon shape, an enneagon, a decagon shape, and a polygon comprising at least eleven sides.

14. The closure device according to claim 12, wherein the carrier tube non-circular cross-section comprises at least one of an elliptical shape, an oval shape, a rectangular shape, a triangle shape, a quadrilateral shape, a pentagon shape, a hexagon shape, a heptagon shape, a octagon shape, an enneagon, a decagon shape, and a polygon comprising at least eleven sides.

15. The closure device according to claim 13, wherein carrier tube non-circular cross-section comprises at least one of an elliptical shape, an oval shape, a rectangular shape, a triangle shape, a quadrilateral shape, a pentagon shape, a hexagon shape, a heptagon shape, a octagon shape, an enneagon, a decagon shape, and a polygon comprising at least eleven sides.

16. The closure device according to claim 15, wherein the carrier tube non-circular cross-section is the same shape as the collagen sponge non-circular cross-section.

17. The closure device according to claim 15, wherein the carrier tube non-circular cross-section is a different shape than the collagen sponge non-circular cross-section.
